(19) United States
(12) Patent Application Publication
(10) Pub. No.: US 2019/0183324 A1
(43) Pub. Date: Jun. 20, 2019

(54) DIGITAL DISPOSABLE ENDOSCOPE SYSTEM

(71) Applicant: Clear Image Technology, LLC, Westlake, OH (US)

(72) Inventor: John Costin, Avon Lake, OH (US)

(21) Appl. No.: 16/041,418

(22) Filed: Jul. 20, 2018

Related U.S. Application Data
(60) Provisional application No. 62/599,203, filed on Dec. 15, 2017.

(51) Int. Cl. A61B 1/06; A61B 1/00; A61B 1/05; A61B 1/005; A61B 1/317

(52) U.S. Cl. CPC A61B 1/0684; A61B 1/00128; A61B 1/05; A61B 2017/0046; A61B 1/005; A61B 1/317; A61B 1/0669

(57) ABSTRACT

A medical imaging system, is formed of a reusable handpiece, including an LED light source, and a connection to an imaging probe. The reusable handpiece can be reused. A disposable imaging probe, is used only once. This has a first portion for connection to the handpiece, a light guide, receiving light from the LED light source, an elongated tube with a first end receiving light from the LED light source, and a second distal end, the tube including a light guide that receives light from the LED light source, and guides the light along the tube to the distal end, and a camera, located at the distal end, facing outward from the distal end, and receiving an image from the distal end, where the camera is surrounded by a light transmissive opening, which illuminates an area around the camera based on light that is transmitted down the LED light source, and imaging an area of the distal end of the handpiece.

This application claims priority from Provisional application No. 62/599,203, filed Dec. 15, 2017; the entire contents of which are herewith incorporated by reference.

### BACKGROUND

Clear Image Technology (CIT), the Applicant of this patent application, has developed an arthroscopic system that includes: (a) a disposable scope, (b) a re-usable hand piece, (c) display/console, and (d) software and image enhancement algorithms. CIT's disposable scope is intended to be a single-use digital arthroscope packaged with a sterile drape. The disposable scope part of this system includes: (a) micro-CMOS camera module (which includes optics) with a ribbon cable style connection, (b) a plastic optical light guide, (c) a stainless steel outer sheath, (d) a printed circuit board embedded in each scope that allows calibration data, (e) electrical contacts to connect with the hand piece, and (g) custom molded plastic parts such as a scope connector and sterile drape cover.

The handpiece contains the following components: (a) a quick-fit connector designed for rapid attachment and removal of the disposable scope (b) custom firmware and electronics for high-speed data processing (c) efficient LED module for providing illumination (d) single-action button for still and video capture and (e) custom molded plastic parts such as the handpiece casing and strain relief.

The display/console is an off-the-shelf part, but it includes (a) a high-definition display, (b) custom software for image processing, procedure data handling, and image capture and (c) a medical grade power supply for increased safety.

The outside diameter of the scope is approximately 2.2 millimeters, which are often called small diameter scopes. There are a number of problems with small-diameter arthroscopic technology prior to ours. These include complicated and bulky equipment being required to operate, heat and noise generated by light sources and fans, difficulty with miniaturizing the scope, decreased durability with small diameter solutions, complicated manufacturing processes, and being too costly to dispose. Other technical concerns include overly complex assembly processes, lower quality fiber-optic imaging, and a high cost of goods.

### SUMMARY

The following are points unique to CIT's technology. These make for a novel way to conduct light efficiently in a compact, cost-effective disposable system and to process the image received from the device.

Embodiments describe a disposable, small diameter, low-cost diagnostic imaging probe with image quality drastically improved over fiber-optic systems, and also having improved hand comfort and improved data processing.

Embodiments describe a way to product light and pass that to the tip of the handset.

### BRIEF DESCRIPTION OF THE DRAWINGS

The different figures show different embodiments.

FIG. 1 shows a complete endoscope system;

FIG. 2 shows a side view of the endoscope tube;

FIG. 3 shows a detailed closeup of the end piece, showing the illumination part and the camera;

FIG. 4 shows the handpiece; and

FIG. 5 shows the connector parts and how they attach.

### DETAILED DESCRIPTION

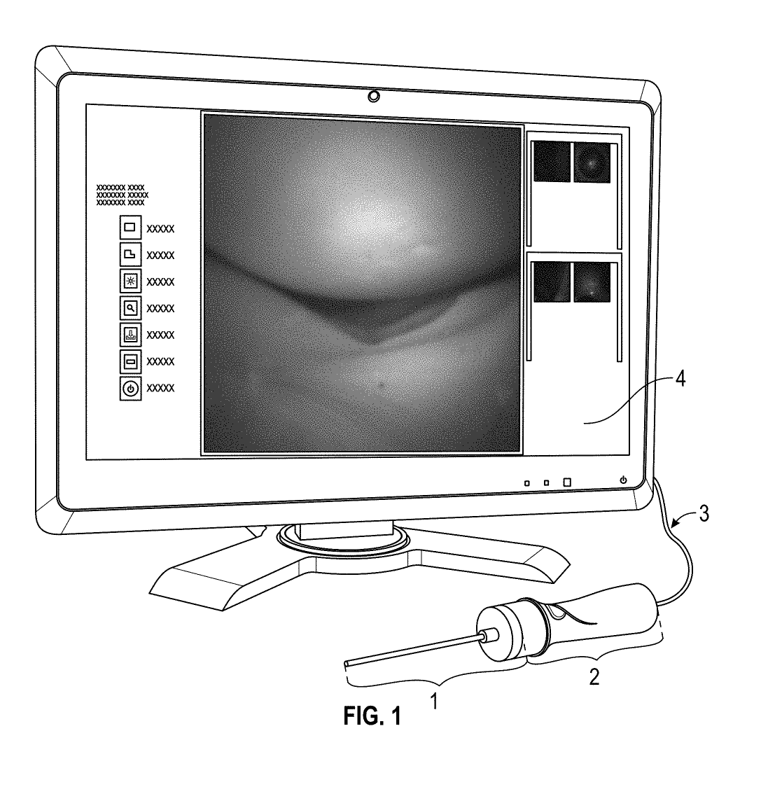

FIG. 1 shows an endoscope system according to an embodiment. The system comprises a disposable imaging probe 1, a reusable handpiece 2 coupled by a connecting connection 3 to a display console 4.

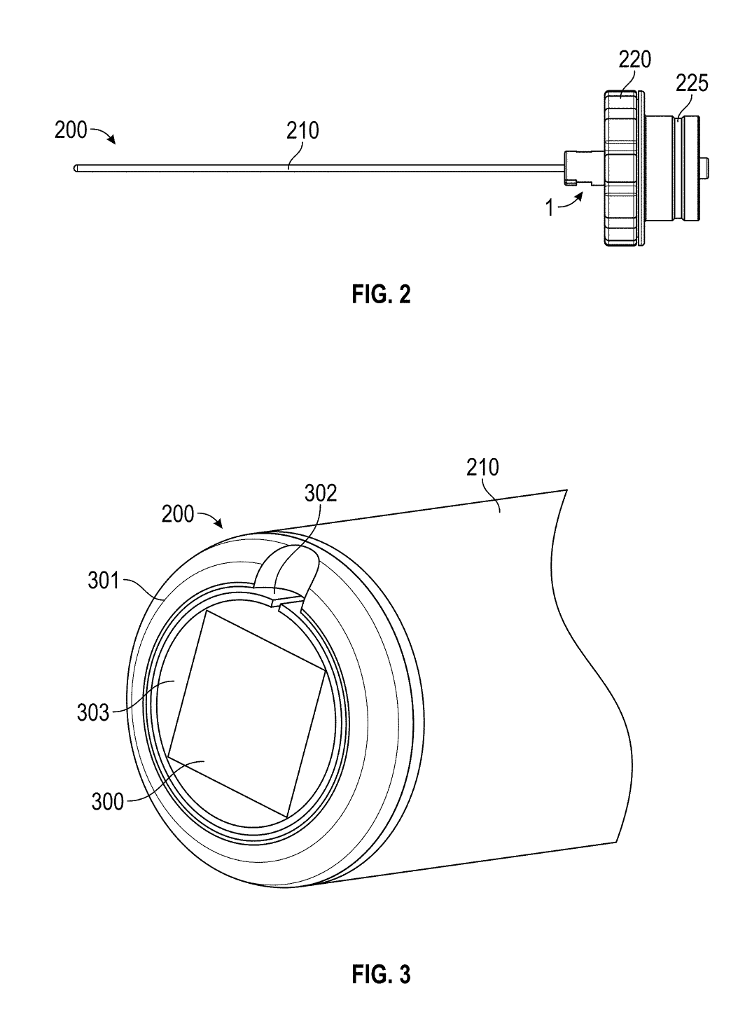

The imaging probe 1 is a separate, detachable, and disposable imaging probe, shown in further detail in FIG. 2. The probe has a camera sensor 200 at its distal tip which is shown in further detail in FIG. 3. The sensor 200 can be a single chip CMOS camera sensor with built in A/D converter and processing in one embodiment. In another embodiment, the sensor 200 can be an ultrasound, OCT, a stereoscopic dual camera sensor, or any combination thereof.

An LED light source can be encapsulated either at the tip, or in the shaft along with the sensor, or an LED source in the handpiece and transmitted down the probe and illuminating the end of the probe. The simplified design of the probe lowers the overall part cost and manufacturing complexity, which minimizes the number of components being disposed of after a procedure.

The device contains a flash chip to store hardware IDs, image correction data, and procedure information to prevent reuse. A scope cover 220 with rubberized gasket 225 creates a seal to prevent fluid ingress into the electronics of the handpiece, when the scope is connected to the handpiece. In one embodiment, the LED is in the handpiece part that is covered by that gasket.

FIG. 3 shows a closeup of the probe tip. The camera sensor 300 is positioned at the distal tip 200 of the probe 210 for direct imaging. The imaging sensor is located at the distal tip of the probe. The proximal portion connects to the handpiece, which contains the light source and electronics.

The imaging sensor 300 is right at the tip of the probe. This compares with other systems which have separation by a complex lens region; or are coherently coupled through an optical fiber bundle. This is important and novel compared to the prior art. In fact, the inventors found that placing the camera directly at the tip means less optical or chromatic distortion due to image transmission through a series of lenses or fibers. Eliminating image transmission through fibers also means no image smoothing is needed to correct fiber-based pixilation, and no “porthole” effect occurs since the sensor is able to directly image the subject instead of imaging the output of a micro-telescope. Another benefit of positioning the sensor at the tip is a drastic reduction in sensitivity to bending or flexing of the scope shaft. The rigidity and alignment of the scope is not critical, since the image will not be distorted by bending or other movement. This also enables modification of the hardware for applications needing a bendable tip, and in one embodiment, the shaft 210 is bendable.

In one embodiment, a wire connects from the handpiece, to the camera, to power the camera and to conduct digital information from the camera to the handpiece for processing.

In another embodiment, the camera is battery operated, using a button style battery, and includes wireless transmission of the data, so no wires are needed. In both embodiments, the light comes from an LED on the handpiece, transmitted down the shaft, and exiting the light guide.

In yet another embodiment, the wireless connection would be from the handpiece to the display, with batteries in the handpiece driving the operation.

There is an indentation or “divot” 302 at the around the sensor aids in assembly of the camera tube into the end of the distal light guide. The light escapes from the entire rounded surface 301, which includes the divot. The camera sensor is in the tip of the probe. In embodiments that use a cable, the cable from the camera runs down the length of the probe.

In an embodiment the sensor is a camera on a chip, including A/D converter, and image processing.

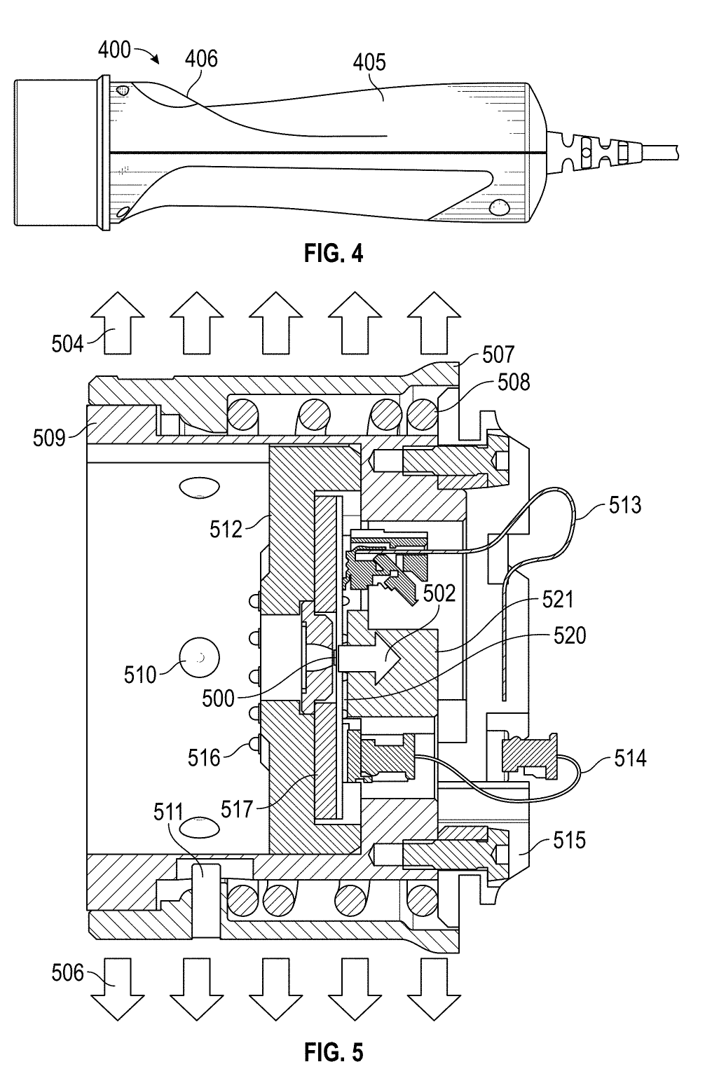

FIG. 4 shows the reusable handpiece part that connects to the probe. The light source and electronics are contained within the handpiece 400, which connects optically, electrically, and mechanically to the detachable disposable imaging probe 1. In one embodiment, the reusable handpiece has a single-button video and still image capture. In an embodiment, the computer decodes the function of the single handpiece button. It can track how often the button is pressed and press duration down to the millisecond. From there, different combinations of pressed and released behavior can be mapped to different software actions. Currently, the button has still image capture mapped to a short (<3 second) press of the button, with video capture mapped to a longer press of the button (>3 seconds).

The handpiece interacts with system operation which decodes button action, allowing a custom button function for different purposes other than stills and videos. In one embodiment, for example, LED power state (on/off) can be mapped to a double press (2 presses within 0.5 seconds).

The handpiece materials and design result in durable hardware, few moving parts, and a shape designed for maximum hand comfort. As shown in FIG. 4, the handpiece includes an outer section with gradually sloping surfaces such as 405 and finger rest sections such as 406. This makes the outer section more comfortable to hold. The handpiece accomplishes comfort in a few main ways. First, the midsection tapers in two dimensions at 406. This creates a resting point for the index finger when holding the handpiece like a remote, or a narrower point to grip when holding like a pencil. The sloping sections also serve as a visual cue on where to hold the hardware—combined with the raised and textured button, the user is encouraged to orient their hand to follow the lines.

The center of balance is near the narrowest point, as well. This allows the surgeon to manipulate the device with minimal effort since they are effectively rotating it around its center of mass. This also allows the button to be pressed without applying a rotational moment and disturbing the image.

The quick-fit connector at the distal end of the handpiece is designed to allow multiple connect/disconnect events and simultaneously manage thermal dissipation. This connector allows easy removal and disposal of the patient-contacting scope while still using high quality materials and sensors by re-using all illumination, heat management, and electronics from procedure to procedure. As the handpiece is not in contact with the patient, it does not need to be sterile, but is designed for effective cleaning.

The handpiece hardware can use the low power consumption of the USB 2.0 protocol, which guarantees low power consumption—5V at up to 0.5 A—to allow for portable visualization via a battery-powered tablet. The USB protocol allows for compatibility with many development and hardware platforms and enables “plug-and-play” usability of the handpiece.

The proximal end of the cable can alternatively be terminated with a variety of custom connectors for additional hardware security.

The handpiece connection serves as both a mechanical coupling for the disposable scope as well as a heat-sink for the LED light source. LED and electronics heat management are integrated with a quick-fit connector, as shown in FIG. 5. This is important and novel because the scope connection hardware is also used as an LED and hardware heat sink. This heat sink, combined with low power draw and efficient construction, makes the system run well below regulatory temperature limits. This further allows optical and electronic hardware to be housed within the handpiece. Combined with the USB connector, this further enables use of any off-the-shelf display console with a standard (here, USB 2.0) port. Putting the LED in the handpiece, where there is a large heatsink, allows use of a more powerful LED die and/or for more current to be delivered to the die.

Image display from the scope and handpiece uses an off the-shelf viewing console loaded with the custom software, which secures patient data access against tampering through drive encryption and a password-protected access. Custom firmware can be used to interface with a variety of off-the-shelf camera hardware, including the sensor at the tip of the scope. Software on the viewing console performs basic image processing, including vignette correction, color correction, and scaling. The same custom software allows saving of stills and video, review and export of past procedure data. The combined benefits of these separate components enable the construction of a disposable, small diameter, low-cost diagnostic imaging probe with image quality drastically improved over fiber-optic systems and small-diameter glass rod scopes.

FIG. 5 shows a detail of the handpiece and its LED module with the two pieces, of the quick connector attached to one another. The inner shell 550 of the quick connect is connected to the reusable part, and the outer shell 555 connects the the disposable part. The LED chip 500 is mounted in a way that it connects to all parts of the connector shell, causing heat to be exhausted through the connector shell as 502, 504 and 506 through both parts of the quick connector. By exhausting the heat in this way, a more robust system can be obtained.

The LED die 500 sits on top of the inner ring 509 of the quick fit connector. There is thermal paste 520 between the LED and a structural block 521, increase thermal flow in the direction 502. The inner ring also routes heat to the outer ring 525 of the connector, where it is radiated to the environment as 504, 506.

The scope is connected to the probe using a three step process. The outer ring 507 (exterior surface) is pulled back, and metal beads 510 sink into a groove created the withdrawing of the ring. The scope is inserted, with a focus on aligning the scope housing with the interior of the connector. Finally, the outer ring of the connector is released. At this time, the metal balls 508 snap into place as the outer ring returns into its default position.

The outer sleeve 507 is mounted over a spring (508) which applies an axial force toward the distal end of the probe. The metal beads 510 protrude through the interior surface of the inner socket 509 and rest in a groove on the probe. When the connector is in the extended, or closed, position, these beads 510 are held firmly in place by a contact surface on the outer sleeve and the probe cannot move. When the sleeve is retracted by compressing the spring 508 toward the proximal end, the beads are released and the probe can be inserted or removed.

A retaining ring 515 is fixed to the proximal end of the inner socket 509 to hold the spring in place. A dowel pin 511 mounted in the outer sleeve 507 makes contact with stops in the inner socket 509 to limit the motion of the sleeve and spring in the proximal and distal directions.

Pogo pins 516 on the inner connector transfer power and data to and from the disposable scope part that is connected to the outer connector. Through these pins, the firmware can get camera data and flash information.

The LED is mounted on a PCB 517 which is mounted at the proximal base of the inner socket 509. The wires 513, 514 connect the LED and probe to the main PCB in the handpiece, although there can be fewer wires in an embodiment. The handpiece contains the processing chips and USB interface hardware.

In operation, Light from the LED 500 is transmitted down the sensor tube 210, through a plastic optical fiber and light guide that runs the entire length of the scope. The optical fiber made of a flexible polymer, and as long as it is not bent past its critical bending radius it can reliably transmit light when bent. Optical fibers provide total internal reflectance, passing the light efficiently down the tube. The fiber can be bent to a small radius without light leaking out the lateral sides. So light is passed down the tube, and illuminates around the edges of the distal end of the probe. The camera images that same area.

Although only a few embodiments have been disclosed in detail above, other embodiments are possible and the inventors intend these to be encompassed within this specification. The previous description of the disclosed exemplary embodiments is provided to enable any person skilled in the art to make or use the present invention. Various modifications to these exemplary embodiments will be readily apparent to those skilled in the art, and the generic principles defined herein may be applied to other embodiments without departing from the spirit or scope of the invention. Thus, the present invention is not intended to be limited to the embodiments shown herein but is to be accorded the widest scope consistent with the principles and novel features disclosed herein.

What is claimed is:

1. A medical imaging system, comprising:
a reusable handpiece, including an LED light source, and a connection to an imaging probe; and
a disposable imaging probe, having a first portion for connection to the handpiece, a light guide, receiving light from the LED light source, an elongated tube with a first end receiving light from the LED light source, and a second distal end, the tube including a light guide that receives light from the LED light source, and guides the light along the tube to the distal end, and
a camera, located at the distal end, facing outward from the distal end, and receiving an image from the distal end, where the camera is surrounded by a light transmissive opening, which illuminates an area around the camera based on light that is transmitted down the LED light source, and imaging an area of the distal end of the handpiece.

2. The medical imaging system as in claim 1, wherein the elongated tube is bendable.

3. The system as in claim 1, wherein the handpiece and the imaging probe connect using a quick connect connector, and where heat from the LED is exhausted through both the disposable imaging probe portion of the quick connect connector, and the reusable handpiece portion of the quick connect connector.

4. The system as in claim 1, wherein the disposable imaging probe portion of the quick connect connector fits inside the reusable handpiece portion of the quick connect connector.

5. The system as in claim 3, wherein the LED is physically inside the perimeter defined by the reusable handpiece portion of the quick connect connector.

6. The system as in claim 5, wherein the distal end of the imaging probe includes a cylindrical surface which emits light received from the LED, and a camera surface, within the cylindrical surface, receiving an image illuminated by the light received from the LED.

7. The system as in claim 4, wherein the imaging probe portion of the quick connect connector has metal balls which snap into place in a seam on an outside of the housing.

8. An endoscope imaging system, comprising:
a reusable handpiece, including a light source, and a first connection portion; and
a disposable imaging probe, having a second connection portion,
where the first connection portion and the second connection portion fit one over the other and snap into place with one connection portion over the other,
where the light source is physically inside an area where the one connection portion fits over the other connection portion;
a light guide, in the disposable imaging probe, and optically coupled to receive light from the light source,
an elongated tube covering at least part of the light guide, with a first end adjacent the reusable handpiece, and a second distal end,
a camera, located at the distal end, facing outward from the distal end, and receiving an image received into the distal end,
where the camera is surrounded by a light transmissive opening in communication with the light guide, which illuminates an area around the camera based on light that is transmitted through the light guide.

9. The medical imaging system as in claim 8, wherein the elongated tube and the light guide are bendable.

10. The system as in claim 8, wherein heat from the light source is exhausted through both the disposable imaging probe portion of the quick connect connector, and the reusable handpiece portion of the quick connect connector.

11. The system as in claim 10, wherein the light source is an LED.

12. The system as in claim 8, wherein the disposable imaging probe portion of the quick connect connector fits inside the reusable handpiece portion of the quick connect connector.

13. The system as in claim 12, wherein the imaging probe portion of the quick connect connector has metal balls which snap into place in a seam on an outside of the housing.

14. A method of imaging an area, comprising:
using a reusable handpiece, to produce light from an LED light source;
attaching a disposable imaging probe, to the reusable handpiece,
the disposable imaging probe having a first portion for connection to the handpiece,
using a light guide in the disposable imaging probe, for receiving light from the LED light source, with an elongated tube with a first end receiving light from the LED light source, and a second distal end, the tube including a light guide that receives light from the LED light source, and guides the light along the tube to the distal end, and
using a camera, located at the distal end, facing outward from the distal end, for receiving an image from the distal end, where the camera is surrounded by a light transmissive opening, which illuminates an area around the camera based on light that is transmitted down the LED light source, and imaging an area of the distal end of the handpiece.

15. The method as in claim 14, wherein the elongated tube is bendable.

16. The method as in claim 14, wherein the handpiece and the imaging probe connect using a quick connect connector, and where heat from the LED is exhausted through both the disposable imaging probe portion of the quick connect connector, and the reusable handpiece portion of the quick connect connector.

17. The method as in claim 14, wherein the disposable imaging probe portion of the quick connect connector fits inside the reusable handpiece portion of the quick connect connector.

18. The method as in claim 16, wherein the LED is physically inside the perimeter defined by the reusable handpiece portion of the quick connect connector.

19. The method as in claim 18, wherein, wherein the distal end of the imaging probe includes a cylindrical surface which emits light received from the LED, and a camera surface, within the cylindrical surface, receiving an image illuminated by the light received from the LED.

20. The method as in claim 14, further comprising, using the disposable imaging probe portion once, and discarding the disposable imaging probe portion after use, and reusing the reusable handpiece with a new the disposable imaging probe portion.
